(19) United States
(12) Patent Application Publication
(10) Pub. No.: US 2004/0175411 A1
(43) Pub. Date: Sep. 9, 2004

(54) Method of treating influenza

(76) Inventor: Arthur Vanmoor, Boca Raton, FL (US)

Correspondence Address: OTTO S KAUDER, 5382C Venetia Ct., Boynton Beach, FL, 33437, US

(21) Appl. No.: 10/378,530

(22) Filed: Mar. 3, 2003

(51) Int. Cl. A61K 31/198; A61K 47/00

(52) U.S. Cl. 424/439; 514/561; 514/563

(57) ABSTRACT

There is disclosed a method of treating influenza in a person in need of such treatment, which comprises the administration to such person of an effective amount of at least one water-soluble aminoacid represented by formula (I)

X—CₙH₂ₙ—CR(NH₂)COOH (I)

wherein X represents a hydrogen atom (H) or a carbamoyl group (CONH₂), R is hydrogen or a methyl (CH₃) group, and n is a whole number from 1 to 4.

### BACKGROUND OF THE INVENTION

1. Field of the Invention

This invention relates to a method of treating a person suffering from influenza with an agent that enhances the effectiveness of the human immune system to mitigate and where possible eliminate the effects of influenza.

2. Description of Related Art

The human immune system functions to maintain human individuality by fighting off foreign entities. The MERCK MANUAL, 16^{th} edition, published 1992, at pages 279 to 303, which portion is here incorporated by reference, contains a detailed description of the parts of the immune system and of immunodeficiency diseases and hypersensitivity disorders to which it is subject. A table at pages 284-5 titled “Cytokines” lists the major effects of such cytokines or immunoeffective polypeptides as interleukin types, interferon types, alpha- and beta-tumor necrosis factor, three types of colony-stimulating factor, and alpha- and beta-transforming growth factor. A table at page 303 lists disorders with increased susceptibility to unusual infections. Nothing in this publication relates to an influenza condition or remedies therefor.

As is well known, remedies for influenza have been sought for generations by a great variety of methods, and with the increasing application of science some successes have been achieved, particularly in the area of vaccines. However, the microorganisms responsible for the disease are known to mutate rapidly, thus giving rise to new strains requiring the development of new vaccines. Moreover, the search for better remedies for this as well as other suffering conditions is enormously costly. For economic reasons, moreover, the search tends to be skewed in the direction of finding novel remedies proprietary to their discoverers and owners. Novel remedies, of course, come into being with nothing known about either their safety or their effectiveness, so that both of these essential attributes need to be exhaustively studied before they can be used as intended.

In contrast, the art has tended to neglect the exploration of therapeutic properties of known substances that humans have been safely ingesting for untold generations. Along these lines, the present inventor has been able to bring about in susceptible individuals within a limited and reproducible time the appearance of headache, elevated blood pressure, facial pimples, signs of the so-called common cold, and pains in a joint by administering selected foods, food ingredients, and relatively harmless household chemicals as trigger substances, and to use these as research tools to study the effectiveness of certain nutrient substances in relieving these artificially produced conditions as well as their natural counterparts. As a result, certain water soluble amino carboxylic acid compounds are disclosed in U.S. Pat. No. ______ as effective against facial pimples; certain water soluble amino carboxylic acid compounds are disclosed in U.S. Pat. No. 5,626,831 as effective against the common cold; certain water soluble amino carboxylic acid compounds are disclosed in U.S. Pat. No. 5,707,967 as effective against headache; certain water soluble amino carboxylic acid compounds are disclosed in U.S. Pat. No. 5,708,029 as effective against elevated blood pressure, and certain water soluble amino carboxylic acid compounds are, disclosed in U.S. Pat. No. 5,767,157 as effective against pain in a joint.

Published PCT application PCT/US01/00714 discloses treating influenza with an aliphatic sulfur compound.

Barrett et al WO 98/52545 discloses a pharmaceutical composition containing the non-steroidal anti-inflammatory drug flurbiprofen and use of the composition in the treatment of the symptoms of colds and flu, particularly sore throat (see page 1 lines 1-6), and, to (see page 1 lines 1-6), and, to overcome an expected unpleasant burning sensation at the back of the mouth caused by fluorbiprofen,

“the combination of a therapeutically effective amount of flurbiprofen with a therapeutically effective amount of one or more active ingredients selected from an antihistamine, a cough suppressant, a decongestant, an expectorant, a muscle relaxant, a centrally acting analgesic, a local anaesthetic, an antibacterial compound, an antiviral compound, an antibiotic compound, an antifungal compound, minerals and vitamins in the form of a masticable or suckable solid dosage form or a liquid or a spray.” (see page 1 line 21 to page 2 line 2).

Barrett further teaches that “suitable expectorant include acetylcysteine, ammonium chloride, carbocysteine, guaifensin and potassium citrate.” (page 2 lines 11-12).

There is no teaching in Barrett et al that any member of the thirteen categories of active ingredients is effective against colds and flu in the absence of a therapeutically effective amount of flurbiprofen.

### SUMMARY OF THE INVENTION

In accordance with this invention, there is provided a method of treating influenza in a person in need thereof, which comprises the administration to such person of an amount of a water-soluble aminoacid represented by formula (I) shown below, effective in mitigating that disease.

X—CₙH₂ₙ—CR(NH₂)COOH (I)

In formula (I), X represents a hydrogen atom (H) or a carbamoyl group (CONH₂), R is hydrogen or a methyl (CH₃) group, and n is a whole number from 1 to 4. When n is greater than 1, the alkylene group represented by CnH₂ₙ can be straight chain or branched.

Without intending to be bound by any theory, the present invention is believed to be beneficial in augmenting the person's innate ability to eliminate toxins from the body and to resist the initiation of the process that makes a person susceptible to influenza as well as to slow down, arrest, and even reverse that process. As a result, the incidence of influenza and the associated discomfort is diminished, and the quality of life is improved.

In mitigating influenza mega-nutrient doses of 2 to 20 grams of a water-soluble aminoacid represented by formula (I) can be administered from one to five times daily, for a total of 2 to 100 grams per day, until monitoring shows sufficient improvement in the user's condition to permit reduction in dose level and ultimately cessation of the treatment. Daily doses of a water-soluble aminoacid represented by formula (I) in the range from 2 to 40 grams per day are preferred. Such doses can be administered in any convenient manner, as by oral administration in any of the usual dosage forms, such as tablets, capsules, solutions, and dispersions in liquid foods such as soups and fruit juices. Alternatively, there can be given sterile solutions of a water-soluble aminoacid represented by formula (I) by direct injection into the bloodstream of the person to be treated, as well as by rectal suppositories containing a water-soluble aminoacid represented by formula (I).

In formula (I), when n=1 the alkylene group represented by CnH₂, is a methylene group. When n=2, the alkylene group can be ethylene —CH₂CH₂— or ethylidene —CH(CH₃)—. When n=3 and n=4, the alkylene group represented by CnH₂ₙ can have each of the known isomeric configurations of such straight chain and branched alkylene groups.

The following table contains preferred water-soluble amino acids represented by formula (I) according to the invention.

| Index | Name | X | R | n |
|---|---|---|---|---|
| 1 | 2-aminobutanoic acid | H | H | 2 |
| 2 | 2-amino-2-methylpropanoic acid | H | CH₃ | 1 |
| 3 | 2-amino-3-carbamoylpropanoic acid | CONH₂ | H | 1 |
| 4 | 2-amino-4-carbamoylbutanoic acid | CONH₂ | H | 2 |
| 5 | 2-amino-3-methylpentanoic acid | H | H | 4 |
| 6 | 2-amino-4-methylpentanoic acid | H | H | 4 |
| 7 | 2-amino-3-methylbutanoic acid | H | H | 3 |

### EXAMPLE 1

Each person in a group of volunteers (12 women and 8 men) with flu symptoms such as sore throat, stuffy nose, and/or headache was given ten gram portions of composition containing several compounds of formula (I) with each meal and at intervals of 2-3 hours between meals. These persons found relief within a few hours and were free of symptoms within 24-48 hours.

What is claimed is:

1. A method of treating influenza in a person in need of such treatment, which comprises the administration to such person of an effective amount of at least one water-soluble amino acids represented by formula (I)
X—CₙH₂ₙ—CR(NH₂)COOH (I)
wherein X represents a hydrogen atom (H) or a carbamoyl group (CONH₂), R is hydrogen or a methyl (CH₃) group, and n is a whole number from 1 to 4.

2. The method of claim 1, wherein the aminoacid is administered orally with food.

3. The method of claim 1, wherein the aminoacid is administered by injection into the bloodstream.

4. The method of claim 1, wherein the aminoacid is administered by rectal suppository.

5. The method of claim 1, wherein the effective amount of the aminoacid is administered in one to five daily doses, each dose being in the range of 2 to 20 gram.

6. The method of claim 1, wherein the total of said effective amount of the aminoacid administered daily is in the range of 2 to 100 grams.

7. The method of claim 6, wherein the total of said effective amount of the aminoacid administered daily is in the range of 20 to 50 grams.

8. The method of claim 1, wherein said person experiences relief from the effects of said condition.

9. The method of claim 1 wherein after treatment said condition is not detectable.

10. The method of claim 1 wherein the aminoacid is 2-amino-4-carbamoylbutanoic acid.

11. The method of claim 1 wherein the aminoacid is 2-aminobutanoic acid

12 The method of claim 1 wherein the aminoacid is 2-amino-2-methylpropanoic acid

13 The method of claim 1 wherein the aminoacid is 2-amino-3-carbamoylpropanoic acid

14. The method of claim 1 wherein the aminoacid is 2-amino-3-methylpentanoic acid

15. The method of claim 1 wherein the aminoacid is 2-amino-4-methylpentanoic acid

16. The method of claim 1 wherein the aminoacid is 2-amino-3-methylbutanoic acid

17. The method of claim 1 wherein a plurality of water-soluble aminoacids represented by formula (I) is administered.
